(19) United States
(12) Patent Application Publication
(10) Pub. No.: US 2024/0198006 A1
(43) Pub. Date: Jun. 20, 2024

(54) Ergonomic Gripping Adaptor for Hypodermic Syringe

(71) Applicant: Lawrence Groop, Las Vegas, NV (US)

(72) Inventor: Lawrence Groop, Las Vegas, NV (US)

(21) Appl. No.: 18/083,564

(22) Filed: Dec. 18, 2022

(51) Int. Cl. A61M 5/31

(52) U.S. Cl. CPC A61M 5/3137; A61M 2205/586

(57) ABSTRACT

An ergonomic gripping adaptor for a hypodermic syringe comprises a size-adjustable thumb ring and size-adjustable rings vertically positioned for the index and middle fingers. The thumb ring is removably attachable to the plunger flange of a standard hypodermic syringe and the finger rings are removably attachable to the barrel flange of the syringe.

### TECHNICAL FIELD OF THE INVENTION

The disclosed device relates to effective and safe injection or application of treatment liquids from a syringe, particularly those for use on skin surfaces. Further embodiments include components that enhance the comfort and efficiency of injecting liquids from a syringe.

### BACKGROUND OF THE INVENTION

Currently, when clinicians inject into a patient's skin with a hypodermic needle, without the aid of molded finger rings attached to the plunger and syringe body, it is not possible to aspirate or pull back on the plunger with the same hand. Aspiration is an important step in injections as it ensures that the liquid is not being unintentionally injected into a blood vessel.

There have been several designs developed to allow a clinician to aspirate. These designs include placing a ring attached to the underside (patient facing side) of the syringe support, one for the index finger and one for the ring finger. Additionally, a ring is affixed to the plunger so that the clinician may put his/her thumb in the plunger ring, his/her index finger in the index ring and his/her middle finger in the middle ring. Once gripped as such, the clinician may now aspirate by pulling back with his/her thumb whilst this movement of the plunger is anchored on the syringe body by the index and middle finger resisting such movement. This allows the plunger to ascend in the syringe and by doing so, draws liquid into the syringe from inside the patient's body.

The problem with this arrangement of finger rings is that, in order to properly grip and inject the patient, the clinician must rotate his/her wrist forward and down. In doing so, the thumb's range of motion, by virtue of the grip angle, is reduced as compared to the thumb's range of motion if the wrist were not rotated down.

This invention comprises an ergonomic gripping adaptor apparatus that changes the position of the index and middle finger ring by placing them on the distal side of the syringe body, stacked vertically. By doing so, when the clinician places his/her index and middle fingers into the rings to inject, it is no longer necessary for the clinician to rotate his/her wrist down. As such, the clinician now has a longer range of motion with the thumb, which is more comfortable, safer, and allows better visibility of the volume markers on the syringe. The stability of this apparatus is enhanced by the addition of a molded syringe guide support which vertically traverses the length of the syringe body. Additionally, a curved finger flange is molded onto the end of the syringe guide support to provide a safe stop so that the clinician's little finger has a guard that keeps it away from the needle and injection site.

Furthermore, as the size of a clinician's finger varies, each finger/thumb ring is adjustable by the utilization of two semi-circles forming the ring and contacting each other via angled teeth. Then the clinician squeezes the two semi-circles together, the teeth ratchet and decrease the diameter of the circle formed.

Unlike other inventions, both the plunger attachment and the syringe body attachment may be attached or removed to a standard barrel syringe of varying size and volumes without the need for a customized syringe, as is the case with prior inventions.

### SUMMARY OF THE INVENTION

A three ring syringe support apparatus is provided that attaches to a syringe, which may be a standard barrel syringe, with a thumb ring that attaches to the plunger, and an index finger ring and middle finger ring that attach to one side of the syringe body where a syringe guide support extends vertically down the length of the syringe.

Both index finger and middle finger rings are placed vertically in relation to each other and the rings are composed of semi-circle extensions with teeth. When squeezed together, the teeth engage and decrease the diameter of the finger hole. At the distal end of the syringe guide support, a curved finger flange protects the little finger of the hand. There is an identically structured thumb ring that is affixed to the plunger. Both plunger and syringe body apparatus may be affixed to standard sized syringes of varying volume.

In one embodiment, the thumb ring and finger ring components are made of metal, such as a thin aluminum alloy, allowing them to be autoclave sterilized and reused.

In another embodiment, the thumb ring and finger ring components are made of plastic designed for single use.

### BRIEF DESCRIPTION OF THE DRAWINGS

To provide a more complete understanding of the present invention and features and advantages thereof, reference is made to the following description, taken in conjunction with the accompanying figures, wherein like reference numerals represent like parts.

FIG. 1 is a side view of an embodiment of the adaptor assembly attached to a barrel syringe.

FIG. 2A is a bottom view of the thumb ring portion of the embodiment of FIG. 1 in an open position before attachment to the syringe.

FIG. 2B is a bottom view of the thumb ring of FIG. 2A in a closed position securing the thumb ring to the syringe.

FIG. 3 is a side view of the operator holding apparatus in his/her hand with thumb inserted into thumb ring, index finger inserted into index finger ring, and middle finger inserted into middle finger ring.

FIG. 4 is a side view of a different embodiment of the invention.

### DETAILED DESCRIPTION

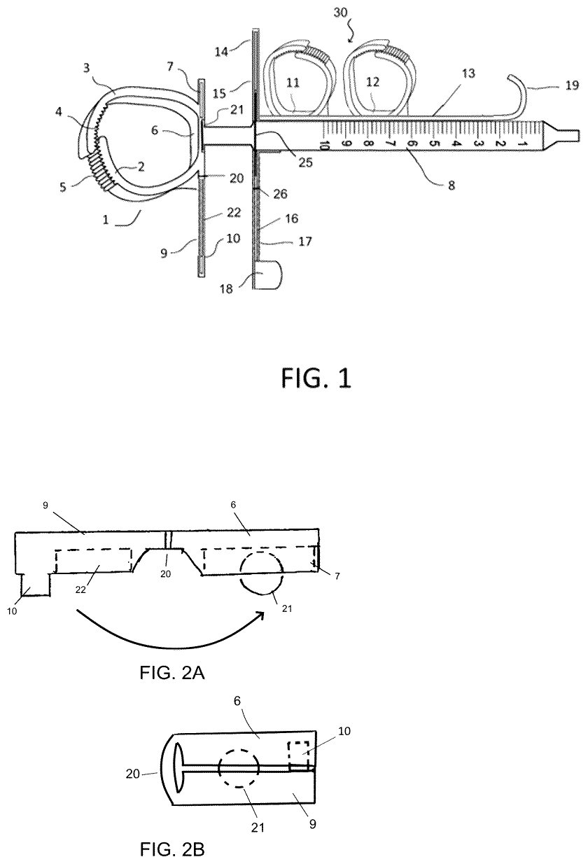

In an embodiment shown in FIG. 1, a gripping adaptor comprises thumb ring component 1 and finger ring component 30. Adjustable thumb ring 1 is a generally circular ring split at the top into two semicircular elements, left semicircle element 2 and right semicircle element 3. The inside surface of right semicircle element 3 has thumb size adjustment teeth 4. The outside surface of left semicircle element 2 has thumb size adjustment teeth 5 that interlock with teeth 4 on right semicircle element 3, allowing the operator to adjust the diameter of the thumb opening by moving left semicircle element 2 under and past right semicircle element 3 and allowing the thumb size adjustment teeth 4 and 5, on both semicircles, to engage at the desired thumb ring diameter. Opposite the split ends, the semicircle elements are connected at the thumb ring support base 6 which includes plunger flange insertion slot 7 and is attached to thumb ring locking arm 9.

As illustrated in FIGS. 2A and 2B, the plunger flange 21 of syringe 8 is placed into plunger flange insertion slot 7. The user then folds thumb ring locking arm 9 at hinge 20 to cover the exposed edge of plunger flange 21 with plunger flange retainer slot 22 and inserts locking tab 10 into the far edge of plunger flange insertion slot 7. Locking tab 10 is slightly thicker than plunger flange insertion slot 7, so that the resulting friction of locking tab 10 inside plunger flange insertion slot 7 keeps thumb ring locking arm 9 fastened to thumb ring support base 6.

Adjustable index finger ring 11 and adjustable middle finger ring 12 are attached to syringe guide support 13, which is an extension that runs down the side of syringe 8. Both adjustable index finger ring 11 and adjustable middle finger ring 12 have the same design as adjustable thumb ring 1, except that these two finger rings have a smaller initial diameter compared to the initial diameter of adjustable thumb ring 1.

The proximal end of syringe guide support 13 is attached to finger support base 14. Finger support base 14 has a barrel flange insertion slot 15 to accept the barrel flange 25 of syringe 8 and is attached to finger support locking arm 17, which includes hinge 26, barrel flange retainer slot 16 and locking tab 18. The distal end of syringe support guide 13 may terminate just past the attachment point of middle finger ring 12 or may optionally extend to form a curved finger flange 19.

To attach finger ring component 30, barrel flange 25 is inserted into barrel flange insertion slot 15 of finger support base 14. Then finger support locking arm 17 is folded at hinge 26 to cover the exposed edge of barrel flange 25 with barrel flange retainer slot 16 while ensuring that locking tab 18 on finger support locking arm 17 slides inside the far edge of barrel flange insertion slot 15. This is the same process as attachment of the thumb ring component described above. Once locking tab 18 is inserted, finger support locking arm 17 is now fastened to finger support base 14 and finger ring component 30 is secured to syringe 8.

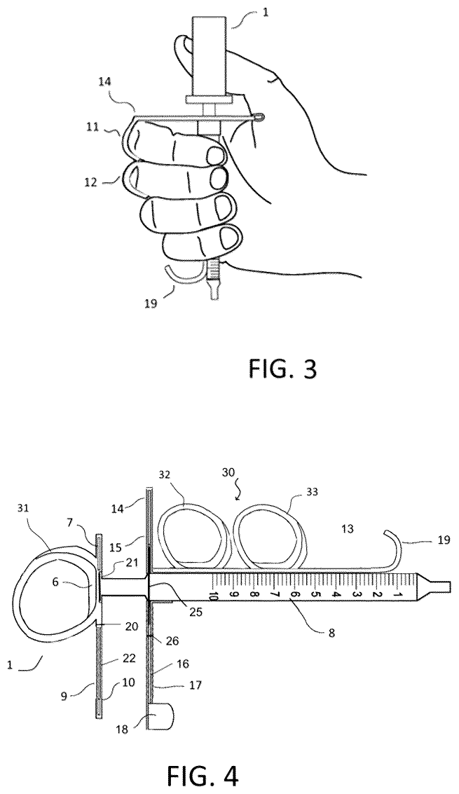

The user can now grasp syringe 8 in an overhand grip with the thumb ring facing up. The operator adjusts the diameter of the thumb ring, the index finger ring and the middle finger ring as described prior and the curved finger flange 19 provides comfortable adaptation to the user's little finger preventing the little finger from coming into contact with the needle. The operator can now comfortably and safely aspirate and inject with an improved, ergonomically supported wrist position and grip on syringe 8. See FIG. 3.

In some circumstances it may be advantageous to implement an adaptor that has fixed ring circumferences rather than variable circumferences. Particularly in the case of autoclavable adaptors, a fixed ring can be made of a more solid and durable metal than that needed to enable a variable circumference. FIG. 4 shows a fixed ring embodiment. The elements are the same as in FIG. 1, except that the thumb ring 31 and the two finger rings 32 and 33 are solid rings of a fixed circumference.

Barrel syringes come in a variety of configurations. In the most common version, the plunger flange (sometimes called the thumb rest) is a circular disc while the barrel flange (sometimes called the finger flange) is an oblong disc. In such case, the finger ring component will be attached in a predetermined orientation controlled by the shape of the flange, whereas the thumb ring component may be turned in any direction before securing it to the circular plunger flange. This permits the user to orient the ring directions for a comfortable grip by attaching the finger ring component first.

Both components of the disclosed adaptor attach by folding a slotted locking arm over a slotted support arm with a flange captured between them. This requires that the components be hinge connected. In the case of a disposable plastic adaptor, the hinge connection may be formed by including a thinner section of plastic between the support arm and the locking arm. In the case of an autoclavable metal adaptor, a mechanical hinge connector may be interposed between the two arms.

The foregoing description has been presented and is intended for the purposes of illustration and description. It is not intended to be exhaustive nor limit the invention to the above teachings. The embodiments were chosen and described in order to best explain the principles of the invention and its practical application and to enable others skilled in the art to best utilize the invention in various embodiments and with various modifications as are suited to the particular use contemplated. Therefore, it is intended that the invention not be limited to the particular embodiments disclosed for carrying out the invention.

I claim:

1. A gripping adaptor for a barrel syringe with a plunger flange and a barrel flange, comprising
a size-adjustable thumb ring removably attachable to the plunger flange;
a syringe guide support removably attachable to the barrel flange and extending partway down the length of the barrel;
a first size-adjustable finger ring attached to the guide support below the barrel flange; and
a second size-adjustable finger ring attached to the guide support below the first finger ring.

2. The adaptor of claim 1 wherein the thumb ring is attached to a thumb ring support base with a plunger flange insertion slot;
the thumb ring support base is hinge connected to a thumb ring locking arm with a plunger flange retainer slot and a first locking tab;
the syringe guide support is attached to a finger support base with a barrel flange insertion slot; and
the finger support base is hinge connected to a finger support locking arm with a barrel flange retainer slot and a second locking tab.

3. The adaptor of claim 2 wherein
the thumb ring comprises first and second semi-circular elements open at an end opposite the thumb ring support base, the first element includes teeth on an outer surface and the second element includes teeth on an inner surface so that the teeth interlock when pressed together, fixing the size of the thumb ring;
the first finger ring comprises first and second semi-circular elements open at an end opposite the syringe guide support, the first element includes teeth on an outer surface and the second element includes teeth on an inner surface so that the teeth interlock when pressed together, fixing the size of the first finger ring; and
the second finger ring comprises first and second semi-circular elements open at an end opposite the syringe guide support, the first element includes teeth on an outer surface and the second element includes teeth on an inner surface so that the teeth interlock when pressed together, fixing the size of the second finger ring.

4. The adaptor of claim 2 wherein the syringe guide support extends nearly the length of the syringe barrel and includes an upwardly curved end to support and protect the user's little finger.

5. The adaptor of claim 3 wherein the syringe guide support extends nearly the length of the syringe barrel and includes an upwardly curved end to support and protect the user's little finger.

6. A gripping adaptor for a barrel syringe with a plunger flange and a barrel flange, comprising
a thumb ring removably attachable to the plunger flange;
a syringe guide support removably attachable to the barrel flange and extending partway down the length of the barrel;
a first finger ring attached to the guide support below the barrel flange; and
a second finger ring attached to the guide support below the first finger ring.

7. The adaptor of claim 6 wherein the thumb ring is attached to a thumb ring support base with a plunger flange insertion slot;
the thumb ring support base is hinge connected to a thumb ring locking arm with a plunger flange retainer slot and a first locking tab;
the syringe guide support is attached to a finger support base with a barrel flange insertion slot; and
the finger support base is hinge connected to a finger support locking arm with a barrel flange retainer slot and a second locking tab.
